(19) United States
(12) Patent Application Publication
(10) Pub. No.: US 2007/0110685 A1
(43) Pub. Date: May 17, 2007

(54) Combination therapy for the treatment of immunoinflammatory disorders

(76) Inventors: Benjamin A. Auspitz, Cambridge, MA (US); Bradley B. Brasher, Natick, MA (US); Todd W. Chappell, Boston, MA (US); Michael G. Frank, Boston, MA (US); Daniel Grau, Arlington, MA (US); Edward Roydon Jost-Price, West Roxbury, MA (US); Seth Lederman, New York, NY (US); Palaniyandi Manivasakam, West Roxbury, MA (US); Seth Orlow, New York, NY (US); Noah Sachs, South Boston, MA (US); Brendan Smith, Somerville, MA (US)

Correspondence Address: CLARK & ELBING LLP, 101 FEDERAL STREET, BOSTON, MA, 02110, US

(21) Appl. No.: 11/454,202

(22) Filed: Jun. 16, 2006

Related U.S. Application Data
(60) Provisional application No. 60/691,766, filed on Jun. 17, 2005.

(51) Int. Cl. A61K 8/49; A61K 38/13; A61K 31/4745; A61K 31/385; A61K 31/522; A61K 31/4178

(52) U.S. Cl. 424/59; 514/11; 514/183; 514/263.32; 514/291; 514/397; 514/471; 424/641

(57) ABSTRACT

The invention features a method for treating a patient diagnosed with, or at risk of developing, an immunoinflammatory disorder by administering a non-steroidal immunophilin-dependent immunosuppressant (NsIDI) and a Group A enhancer (e.g., antifungal agent, antigout agent, anti-infective agent, antiprotozoal agent, antiviral agent, humectant, sunscreen, vitamin D compound, microtubuline inhibitor, or zinc salt) or analog or metabolite thereof to the patient. The invention also features a pharmaceutical composition containing an NsIDI and Group A enhancer or analog or metabolite thereof for the treatment or prevention of an immunoinflammatory disorder.

### CROSS-REFERENCE TO RELATED APPLICATIONS

This application claims benefit of U.S. provisional application No. 60/691,766, filed Jun. 17, 2005, which is incorporated herein by reference.

### BACKGROUND OF THE INVENTION

The invention relates to the treatment of immunoinflammatory disorders.

Immunoinflammatory disorders are characterized by the inappropriate activation of the body's immune defenses. Rather than targeting infectious invaders, the immune response targets and damages the body's own tissues or transplanted tissues. The tissue targeted by the immune system varies with the disorder. For example, in inflammatory dermatoses, the immune response is directed against the skin. Inflammatory dermatoses affect millions of individuals and include conditions such as atopic dermatitis, psoriasis, pyoderma gangrenosum, lichen planus, rosacea, and seborrheic dermatitis. Immunoinflammatory disorders targeting tissues other than the skin include conditions such as asthma, allergic intraocular inflammatory diseases, arthritis, diabetes, hemolytic anaemia, inflammatory bowel or gastrointestinal disorders (e.g., Crohn's disease and ulcerative colitis), multiple sclerosis, myasthenia gravis, pruritis/inflammation, rheumatoid arthritis, cirrhosis, and systemic lupus erythematosus.

Current treatment regimens for immunoinflammatory disorders typically rely on immunosuppressive agents. The effectiveness of these agents can vary and their use is often accompanied by adverse side effects. Thus, improved therapeutic agents and methods for the treatment of immunoinflammatory disorders are needed.

### SUMMARY OF THE INVENTION

We have discovered that a combination of a non-steroidal immunophilin-dependent immunosuppressant (NsIDI) (e.g., cyclosporine A) and a Group A enhancer (e.g., antifungal agent, antigout agent, anti-infective agent, antiprotozoal agent, antiviral agent, humectant, sunscreen, vitamin D compound, or zinc salt) is more effective in suppressing secretion of proinflammatory cytokines than either agent alone. Thus, combinations of an NsIDI and the above agents, as well as their structural or functional analogs, can be used in an anti-immunoinflammatory combination of the invention.

In one aspect, the invention generally features a composition containing an NsIDI and a Group A enhancer in amounts that together are sufficient in vivo to decrease proinflammatory cytokine secretion or production or to treat an immunoinflammatory disorder.

Optionally, the composition further contains a non-steroidal anti-inflammatory drug (NSAID), a COX-2 inhibitor, a biologic, a disease-modifying anti-rheumatic drugs (DMARD), a xanthine, an anticholinergic compound, a beta receptor agonist, a bronchodilator, a corticosteroid, a small molecule immunomodulator, a humectant, a zinc salt, a psoralen, a retinoid, a vitamin D compound, or a 5-amino salicylic acid. In some embodiments, the composition is formulated for topical or systemic administration.

The invention also provides a method of decreasing proinflammatory cytokine secretion or production in a patient by administering to the patient a composition containing an NsIDI and a Group A enhancer in amounts that together are sufficient in vivo to decrease proinflammatory cytokine secretion or production in the patient.

The invention also features a method of decreasing proinflammatory cytokine secretion or production in a patient. The method includes administering to the patient an NsIDI and a Group A enhancer simultaneously or within 14 days of each other in amounts that together are sufficient in vivo to decrease proinflammatory cytokine secretion or production in the patient.

In addition, the invention features a method for treating a patient diagnosed with or at risk of developing an immunoinflammatory disorder. The method includes administering to the patient an NsIDI and a Group A enhancer simultaneously or within 14 days of each other in amounts sufficient to treat the patient. In one embodiment, the NsIDI and the Group A enhancer are administered together in one composition.

The invention also features a method of decreasing proinflammatory cytokine secretion or production in a cell (e.g., a mammalian cell in vivo). The method includes contacting the cell with an NsIDI and a Group A enhancer simultaneously or within 14 days of each other in amounts sufficient in vivo to decrease proinflammatory cytokine secretion or production in the cell.

The invention features a method of treating a patient diagnosed with or at risk of developing proliferative skin disease. The method includes administering to the patient an NsIDI and a Group A enhancer simultaneously or within 14 days of each other in amounts sufficient to treat the patient. In one embodiment, the NsIDI and the Group A enhancer are administered together in one composition.

The invention further provides a kit containing a composition containing an NsIDI and a Group A enhancer, and instructions for administering the composition to a patient diagnosed with or at risk of developing an immunoinflammatory disorder.

The invention also provides a kit containing an NsIDI, a Group A enhancer, and instructions for administering the NsIDI and the Group A enhancer to a patient diagnosed with or at risk of developing an immunoinflammatory disorder.

The invention also provides a kit containing an NsIDI; and instructions for administering the NsIDI and a Group A enhancer to a patient diagnosed with or at risk of developing an immunoinflammatory disorder.

In addition, the invention provides a kit containing a Group A enhancer and instructions for administering the Group A enhancer and an NsIDI to a patient diagnosed with or at risk of developing an immunoinflammatory disorder.

In preferred embodiments of any of the previous aspects, a Group A enhancer is, for example, an antifungal agent, such as clotrimazole; an antigout agent, such as colchicine; an antiviral agent, such as acyclovir; an antiprotozoal agent, such as metronidazole; anti-infective agent, such as nitrofurazone; a sunscreen agent, such as oxybenzone; a humectant, such as urea; a vitamin D compound, a microtubulin inhibitor, or a zinc salt. The Group A enhancer can be selected from any of the Group A enhancers identified herein.

In another preferred embodiment of any of the previous aspects, the NsIDI and the Group A enhancer are formulated for topical administration. The topical formulation can include greater than 0.10, 0.25, 0.5, 1, 2, 3, 4, 5, 6, 7, 8, 9, 10, 15, 20, 25, 30, or even 35% (w/w) zinc. Desirably, the combination is formulated as a cream, foam, paste, lotion, gel, stick, spray, patch, or ointment and applied topically for the treatment of a dermal inflammatory disorder, such as psoriasis, atopic dermatitis, hand dermatitis, or actinic keratosis.

In preferred embodiments of any of the previous aspects, an NsIDI is, for example, a calcineurin inhibitor, such as cyclosporine, tacrolimus, ascomycin, pimecrolimus, ABT-281, or ISAtx247, or a molecule interacting with FK506-binding protein, such as rapamycin or everolimus.

In one embodiment of any of the previous aspects, the therapeutically active ingredients of the combination consist of an NsIDI and a Group A enhancer.

For any combination described herein, the invention features the use of the active ingredients of the combination in the manufacture of a medicament for the treatment of any immunoinflammatory disorder or proliferative skin disease described herein. The medicament can be prepared using any of the formulation techniques described herein. Furthermore, the medicament can be administered using any of the methods described herein.

Preferred combinations of the invention include cyclosporine and acyclovir; tacrolimus and acyclovir; ascomycin and acyclovir; pimecrolimus and acyclovir; ABT-281 and acyclovir; ISAtx247 and acyclovir; rapamycin and acyclovir; everolimus and acyclovir; cyclosporine and clotrimazole; tacrolimus and clotrimazole; ascomycin and clotrimazole; pimecrolimus and clotrimazole; ABT-281 and clotrimazole; ISAtx247 and clotrimazole; rapamycin and clotrimazole; everolimus and clotrimazole; cyclosporine and colchicine; tacrolimus and colchicine; ascomycin and colchicine; pimecrolimus and colchicine; ABT-281 and colchicine; ISAtx247 and colchicine; rapamycin and colchicine; everolimus and colchicine; cyclosporine and metronidazole; tacrolimus and metronidazole; ascomycin and metronidazole; pimecrolimus and metronidazole; ABT-281 and metronidazole; ISAtx247 and metronidazole; rapamycin and metronidazole; everolimus and metronidazole; cyclosporine and nitrofurazone; tacrolimus and nitrofurazone; ascomycin and nitrofurazone; pimecrolimus and nitrofurazone; ABT-281 and nitrofurazone; ISAtx247 and nitrofurazone; rapamycin and nitrofurazone; everolimus and nitrofurazone; cyclosporine and oxybenzone; tacrolimus and oxybenzone; ascomycin and oxybenzone; pimecrolimus and oxybenzone; ABT-281 and oxybenzone; ISAtx247 and oxybenzone; rapamycin and oxybenzone; everolimus and oxybenzone; cyclosporine and urea; tacrolimus and urea; ascomycin and urea; pimecrolimus and urea; ABT-281 and urea; ISAtx247 and urea; rapamycin and urea; everolimus and urea; cyclosporine and a zinc salt; tacrolimus and a zinc salt; ascomycin and a zinc salt; pimecrolimus and a zinc salt; ABT-281 and a zinc salt; ISAtx247 and a zinc salt; rapamycin and a zinc salt; everolimus and a zinc salt; cyclosporine and vitamin D2; tacrolimus and vitamin D2; ascomycin and vitamin D2; pimecrolimus and vitamin D2; ABT-281 and vitamin D2; ISAtx247 and vitamin D2; rapamycin and vitamin D2; everolimus and vitamin D2; cyclosporine and vitamin D3; tacrolimus and vitamin D3; ascomycin and vitamin D3; pimecrolimus and vitamin D3; ABT-281 and vitamin D3; ISAtx247 and vitamin D3; rapamycin and vitamin D3; everolimus and vitamin D3; and any of the preceding combinations further including urea, pantothenol, or a zinc salt.

In certain embodiments of the compositions, kits, and methods of the invention, the only pharmacologically active agents in the composition or kit, or used in the method, are those recited (e.g., NsIDI and a Group A enhancer or NsIDI, a Group A enhancer, and an additional recited agent). In this embodiment, pharmacologically inactive excipients may also be present in the composition or kit, or used in the practice of the method.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, ABT-281, or ISAtx247, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “Group A enhancer” is meant an antiviral agent, antifungal agent, antigout agent, antiprotozoal agent, anti-infective agent, sunscreen agent, microtubule inhibitor, humectant, vitamin D compound, or zinc salt.

By “corticosteroid” is meant any naturally occurring or synthetic compound characterized by a hydrogenated cyclopentanoperhydrophenanthrene ring system and having immunosuppressive and/or anti-inflammatory activity. Naturally occurring corticosteriods are generally produced by the adrenal cortex. Synthetic corticosteriods may be halogenated. Examples of corticosteroids are provided herein.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharmaceuticals).

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant the dosage between the low dosage and the high dosage.

By “treating” is meant administering or prescribing a pharmaceutical composition for the treatment or prevention of an immunoinflammatory disease or proliferative skin disease.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound in the methods, compositions, and kits of the invention, required to treat or prevent an immunoinflammatory disease or proliferative skin disease in a clinically relevant manner. A sufficient amount of an active compound used to practice the present invention for therapeutic treatment of conditions caused by or contributing to an immunoinflammatory disease or proliferative skin disease varies depending upon the manner of administration, the age, body weight, and general health of the patient. Ultimately, the prescribers will decide the appropriate amount and dosage regimen.

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hepatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; focal segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; immune thrombocytopenic purpura inflammatory bowel or gastrointestinal disorders, inflammatory dermatoses; lichen planus; lupus nephritis; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

As used herein, “non-dermal inflammatory disorders” include, for example, rheumatoid arthritis, inflammatory bowel disease, asthma, and chronic obstructive pulmonary disease.

By “dermal inflammatory disorders” or “inflammatory dermatoses” is meant an inflammatory disorder selected from psoriasis, guttate psoriasis, inverse psoriasis, pustular psoriasis, erythrodermic psoriasis, acute febrile neutrophilic dermatosis, eczema, asteatotic eczema, dyshidrotic eczema, vesicular palmoplantar eczema, acne vulgaris, atopic dermatitis, contact dermatitis, allergic contact dermatitis, dermatomyositis, exfoliative dermatitis, hand eczema, pompholyx, rosacea, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, Sweet's disease, neutrophilic hidradenitis, sterile pustulosis, drug eruptions, seborrheic dermatitis, pityriasis rosea, cutaneous kikuchi disease, pruritic urticarial papules and plaques of pregnancy, Stevens-Johnson Syndrome and Toxic Epidermal Necrolysis, tatoo reactions, Wells Syndrome (eosinophilic cellulitis), reactive arthritis (Reiter's Syndrome), bowel-associated dermatosis-arthritis syndrome, rheumatoid neutrophilic dermatosis, neutrophilic eccrine hidradenitis, neutrophilic dermatosis of the dorsal hands, balanitis circumscripta plasmacellularis, balanoposthitis, Behcet's disease, erythema annulare centrifugum, erythema dyschromicum perstans, erythema multiforme, granuloma annulare, hand dermatitis, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both a proliferative skin disease and an inflammatory dermatosis. An example of such a disease is psoriasis.

By “sustained release” or “controlled release” is meant that the therapeutically active component is released from the formulation at a controlled rate such that therapeutically beneficial blood levels (but below toxic levels) of the component are maintained over an extended period of time ranging from e.g., about 12 to about 24 hours, thus providing, for example, a 12 hour or a 24 hour dosage form.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Desirably, the pharmaceutical salt is a zinc salt.

Other features and advantages of the invention will be apparent from the following detailed description, and from the claims.

### DETAILED DESCRIPTION

The invention features methods, compositions, and kits for the treatment of immunoinflammatory disorders by administering an effective amount of a non-steroidal immunophilin-dependent immunosuppressant (NsIDI), such as cyclosporine, and a Group A enhancer (e.g., antifungal agents, antigout agents, anti-infective agents, antiprotozoal agents, antiviral agents, humectants, sunscreens, microtubuline inhibitors, and zinc salts).

The invention is described in greater detail below.

Non-Steroidal Immunophilin-Dependent Immunosuppressants

In one embodiment, the invention features methods, compositions, and kits employing an NsIDI and a Group A enhancer, optionally with a corticosteroid or other agent described herein.

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

Cyclosporines

The cyclosporines are fungal metabolites that comprise a class of cyclic oligopeptides that act as immunosuppressants. Cyclosporine A and its deuterated analogue ISAtx247 are hydrophobic cyclic polypeptides consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL and Sandimmune by Novartis, Gengraf by Abbott, and Restasis by Allergan. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered topically, intravenously, or orally, but topical administration is preferred.

To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL®).

Typically, patient dosage of an oral cyclosporine varies according to the patient's condition, but some standard recommended dosages in prior art treatment regimens are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids. Additional information is provided in Table 1.

TABLE 1
NsIDIs

| | Atopic | | | | | | |
|---|---|---|---|---|---|---|---|
| Compound | Dermatitis | Psoriasis | RA | Crohn's | UC | Transplant | SLE |
| CsA | 0.5-4 | 0.5-4 | 0.5-4 | 6-8 | 6-8 | ˜7-12 | 2.2-6.0 |
| (NEORAL) | mg/kg/day | mg/kg/day | mg/kg/day | mg/kg/day | mg/kg/day | mg/kg/day | mg/kg/day |
| | | | | (oral-fistulizing) | (oral) | | |
| Tacrolimus | .03-0.1% | .05-1.15 | 1-3 | 0.1-0.2 | 0.1-0.2 | 0.1-0.2 | N/A |
| | cream/twice day | mg/kg/day | mg/day | mg/kg/day | mg/kg/day | mg/kg/day | |
| | (30 and | (oral) | (oral) | (oral) | (oral) | (oral) | |
| | 60 gram tubes) | Oleal gel | | | | | |
| Pimecrolimus | 1% | 40-60 | 40-60 | 80-160 | 160-240 | 40-120 | 40-120 |
| | cream/twice day | mg/day | mg/day | mg/day | mg/day | mg/day | mg/day |
| | (15, 30, | (oral) | (oral) | (oral) | (oral) | (oral) | (oral) |
| | 100 gram tubes) | | | | | | |

Legend
CsA = cyclosporine A
RA = rheumatoid arthritis
UC = ulcerative colitis
SLE = systemic lupus erythamatosus

Tacrolimus

Tacrolimus (PROGRAF™, PROTOPIC™, also known as FK506) is an immunosuppressive agent that targets T-cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. It suppresses the immune system and prolongs the survival of transplanted organs. It is currently available in oral and injectable formulations.

Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; 0-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918. All of the tacrolimus analogs described above can be used in place of tacrolimus in the combinations of the invention.

While suggested dosages will vary with a patient's condition, standard recommended dosages used in prior art treatment regimens are provided below. Patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Topical tacrolimus ointment contains either 0.03% or 0.1% of tacrolimus in a base of mineral oil, paraffin, propylene carbonate, white petrolatum and white wax. Patients receiving topical tacrolimus typically receive 0.3% or 0.1% ointment twice daily; and many other formulations are in development. Treatment is often continued for one week after clearing of signs and symptoms.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus. Thus, this metabolite can be used in place of tacrolimus in the combinations of the invention.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is a 33-epi-chloro derivative of the ascomycin. It is produced by the strain Streptomyces hygroscopicus var. ascomyceitus. Like tacrolimus, pimecrolimus (ELIDEL™, Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is particularly useful for the treatment of atopic dermatitis. Pimecrolimus is currently available as a 1% cream. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Each gram of Elidel Cream 1% contains 10 mg of pimecrolimus in a whitish cream base of benzyl alcohol, cetyl alcohol, citric acid, mono- and di-glycerides, oleyl alcohol, propylene glycol, sodium cetostearyl sulphate, sodium hydroxide, stearyl alcohol, triglycerides, and water. Patients receiving topical pimecrolimus typically receive 1% cream twice daily. Combinations of the invention can be formulated in a similar fashion.

Rapamycin

Rapamycin (RAPAMUNE® (sirolimus, Wyeth) is a cyclic lactone produced by Streptomyces hygroscopicus. Rapamycin is an immunosuppressive agent that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A.

Rapamycin is currently available for oral administration in liquid and tablet formulations. RAPAMUNE™ liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1-mg/m²/day maintenance dose is used.

Peptide Moieties

Peptides, peptide mimetics, peptide fragments, either natural, synthetic or chemically modified, that impair the calcineurin-mediated dephosphorylation and nuclear translocation of NFAT are suitable for use in practicing the invention. Examples of peptides that act as calcineurin inhibitors by inhibiting the NFAT activation and the NFAT transcription factor are described, e.g., by Aramburu et al., Science 285:2129-2133, 1999 and Aramburu et al., Mol. Cell 1:627-637, 1998. As a class of calcineurin inhibitors, these agents are useful in the methods of the invention.

Group A Enhancers

The invention features a combination of NsIDI and Group A enhancer or the treatment of immunoinflammatory disorders. Group A enhancers include antifungal agents, antigout agents, anti-infective agents, antiprotozoal agents, antiviral agents, humectants, sunscreens, microtubule inhibitors, vitamin D compounds, and zinc salts.

Antiviral Agents

Antiviral agents which can be used in the combinations of the invention include, without limitation, abacavir, acemannan, acyclovir, adefovir, amantadine, amidinomycin, ampligen, amprenavir, atevirdine, capravirine cidofovir, delavirdine, didanosine, dideoxyadenosine, n-docosanol, edoxudine, efavirenz, emtricitabine, famciclovir, floxuridine, fomivirsen, foscarnet sodium, ganciclovir, idoxuridine, imiquimod, indinavir, inosine pranobex, interferon-α, interferon-β, kethoxal, lamivudine, lopinavir, lysozyme, madu, methisazone, moroxydine, nelfinavir, nevirapine, oseltamivir, palivizumab, penciclovir, enfuvirtide, pleconaril, podophyllotoxin, ribavirin, rimantadine, ritonavir, saquinavir, sorivudine, stallimycin, statolon, stavudine, tenofovir, tremacamra, trifluridine, tromantadine, valacyclovir, valganciclovir, vidarabine, zalcitabine, zanamivir, zidovudine, resiquimod, atazanavir, tipranavir, entecavir, fosamprenavir, merimepodib, docosanol, vx-950, and peg interferon.

One desirable antiviral agent for use in the methods, compositions, and kits of the invention is acyclovir. Acyclovir is used to treat the symptoms of chickenpox, shingles, herpes virus infections of the genitals (sex organs), the skin, the brain, and mucous membranes (lips and mouth), and widespread herpes virus infections in newborns. Acyclovir is also used to prevent recurrent genital herpes infections.

Structural analogs of antiviral agents which may be used in place of acyclovir in the combinations of the invention include, without limitation, 9-((2-aminoethoxy)methyl)guanine, 8-hydroxyacyclovir, 2′-O-glycyl acyclovir, ganciclovir, PD 116124, valacyclovir, omaciclovir, valganciclovir, buciclovir, penciclovir, valmaciclovir, carbovir, theophylline, xanthine, 3-methylguanine, enprofylline, cafaminol, 7-methylxanthine, L 653180, BMS 181164, valomaciclovir stearate, deriphyllin, acyclovir monophosphate, acyclovir diphosphate dimyristoylglycerol, and etofylline.

Acyclovir is currently available in cream, suspension, eye ointment, IV injection, and tablets. Acyclovir is available under the trade name Zovirax. Zovirax tablets are available in 200 mg, 400 mg, and 800 mg formulations. Zovirax cream contains 5% acyclovir. Cream excipients include polxamer 407, cetostearyl alcohol, sodium lauryl sulphate, white soft paraffin, liquid paraffin, propylene glycol and purified water. Combinations of the invention can be formulated in a similar fashion.

For the treatment of herpes simplex infections, Zovirax tablets (200 mg or 400 mg) are typically taken five times daily at approximately four hourly intervals omitting, the night time dose. Treatment generally continues for 5 days, but in severe initial infections may be extended. For treatment of varicella and herpes zoster infections, Zovirax tablets (800 mg) are generally taken five times daily at approximately four-hourly intervals, omitting the night time dose, for seven days. Zovirax Cream is typically applied five times daily at approximately four hourly intervals, omitting the night time application, for 5 days.

Penciclovir is most commonly used to treat herpes simplex viral infections, also known as cold sores. Penciclovir is available in a cream form by the trade name Vectavir or Denavir. Denavir is available for topical administration as a 1% white cream. Each gram of denavir contains 10 mg of penciclovir and the following inactive ingredients: cetomacrogol 1000 BP, cetostearyl alcohol, mineral oil, propylene glycol, purified water and white petrolatum. Denavir cream is generally applied to the affected area at approximately 2 hourly intervals throughout the day for 4 days. Combinations of the invention can be formulated in a similar fashion.

Antifungal Agents

The invention features methods, compositions, and kits that include an antifungal agent (or analog thereof) and NsIDI. The antifungal agent can be from any of a variety of classes of antifungal compounds including, without limitation, amphotericin B (a macrolide polyene that interacts with fungal membrane sterols) flucytosine (a fluoropyrimidine that interferes with fungal protein and DNA biosynthesis) and azoles (e.g., ketoconazole, itraconazole, and fluconazole) which inhibit fungal membrane-sterol biosynthesis, allyolamines, and ciclopirox.

Antifungal agents which can be used in the combinations of the invention include, without limitation, 2-(methoxymethyl)-5-nitrofuran, 2,4,6-tribromo-m-cresol, 3-amino-4-hydroxybutyricacid, acrisorcin, amorolfine, amphotericin, anidulafungin, azaserine, benzalkonium chloride, benzoicacid, bifonazole, biphenamine, bromosalicylchloranilide, buclosamide, butenafine, butoconazole, candicidin, caspofungin, chlordantoin, chlormidazole, chlorphenesin, ciclopirox, ciclopirox olamine, clindamycin, cloconazole, clotrimazole, cloxyquin, coparaffinate, croconazole, dermostatin, diamthazole, diiodohydroxyquinoline, econazole, econozole, enilconazole, er30346, erigeron bonariensisl, erythromycin, exalamide, fenticonazole, filipin, fluconazole, flucytosine, flutrimazole, fungichromin, griseofulvin, griseofulvina, hachimycin, halethazole, haloprogin, hamycin, imazalil, isoconazole, isotretinoin, itraconazole, ketoconazole, lanoconazole, liranaftate, loflucarban, lucensomycin, mepartricin, methylrosanilinium chloride, micafungin, naftifine, natamycin, neomycin undecylenate, neticonazole, nifuratel, nystatin, oligomycin, omoconazole, oxiconazole, oxiconazole nitrate, paraconazole, pecilocin, perimycin, piroctone, posaconazole, propionic acid, pyrithione, pyrrolnitrin, ravuconazole, salicylanilide, saperconazole, sch56592, selenium sulfide, sertaconazole, siccanin, sulbentine, sulconazole, tenonitrozole, terbinafine, terconazole, terfenadine, tioconazole, tolciclate, tolindate, tolnaftate, triacetin, trioxysalen, tubercidin, undecylenate, viridin, voriconazole, and zinoconazole.

One desirable antifungal agent for use in the methods, compositions, and kits of the invention is clotrimazole.

Clotrimazole is used to treat yeast infections of the vagina, mouth, and skin such as athlete's foot, jock itch, and body ringworm. It can also be used to prevent oral thrush in certain patients.

Clotrimazole is sold as a cream, lotion, and solution to apply to the skin; lozenges (called troches) to dissolve in the mouth; and vaginal tablets and vaginal cream to be inserted into the vagina. Clotrimazole is available under the trade name lotrimin. Each gram of lotrimin Cream contains 10 mg clotrimazole, USP in a vanishing cream base of benzyl alcohol NF (1%), cetearyl alcohol 70/30 (10%), cetyl esters wax NF, octyldodecanol NF, polysorbate 60 NF, sorbitan monostearate NF, and purified water USP. Each mL of lotrimin Topical Solution contains 10 mg clotrimazole, USP in a nonaqueous vehicle of PEG 400 NF. Combinations of the invention can be formulated in a similar fashion.

Clotrimazole is usually used five times a day for 14 days for oral thrush, twice a day (in the morning and evening) for 2 to 8 weeks for skin infections, and once a day at bedtime for 3 or 7 days for vaginal infections. The lozenges should be placed in the mouth and dissolved slowly over about 15 to 30 minutes.

Another antifungal agent useful in the methods, compositions, and kits of the invention is cicloprox. Ciclopirox cream (penlac) is sold as an 8% topical solution for nail fungas or loprox shampoo, containing 1% ciclopirox, indicated for the treatment of seborrheic dermatitis of the scalp. Yet another example is econazole (spectazole) available as a 1% topical cream for tinea infections. Another example of antifungal cream is metronidazole (noritate) available as a 1% cream for treatment of rosacea. Yet another example of an antifungal cream is miconazole (monistat) available as a 2% vaginal cream. Another example of an antifungal is terbinafine HCl (lamisil), available as a 1% cream for treatment of athlete's foot.

Antigout Agents

The invention features methods, compositions, and kits that include an antigout agent (or analog thereof) and NsIDI. Antigout are compounds, which are used to treat the disease gout or familial Mediterranean fever.

Antigout agents which can be used in the combinations of the invention include, without limitation, aa 193, allopurinol, benzbromarone, bof 4272, capsaicin, colchicines, etoricoxib, febuxostat, interleukin-1 receptor antagonist, irtemazole, kt 433, oxipurinol, peperomia pellucida, piroxicam, probenecid, rasburicase, sulfinpyrazone, uricase (available from Enzon, Phoenix Pharmacologics, and Savient).

One desirable antigout agent for use in the methods, compositions, and kits of the invention is colchicine, a major alkaloid from Colchicum autumnale L. and found also in other Colchicum species.

Structural analogs of colchicine which may be used in place of colchicine in the combinations of the invention include, without limitation, isocolchicine, colchiceine, colchicine, 3-demethyl-(7ci), 3-desmethylcolchicine, 4-formylcolchicine, colchicide, colchicenamide, isocolchicine, colchicenamide, colchifoline, thiocholchicine, chlorcolchicine, bromocolchicine, and lumicolchicine.

Microtubule Inhibitors

The invention features methods, compositions, and kits that include a microtubule inhibitor (or analog thereof) and NsIDI. Microtubule inhibitors are agents that affect the equilibrium between free tubulin dimers and assembled polymers.

Microtubule inhibitors which can be used in the combinations of the invention include, without limitation, colchicine, docetaxel, paclitaxel, podofilotoxin, podofilox, and vinca alkaloids (e.g., vinblastine, vincristine, vinorelbine, and vindesine).

Antiprotozoal Agents

The invention features methods, compositions, and kits that include an antiprotozoal agent (or analog thereof) and NsIDI.

Antiprotozoal agents which can be used in the combinations of the invention include, without limitation, acetarsol, acetarsone, acranil, aminitrozole, anisomycin, antimony, azanidazole, benznidazole, berberine, chloroquine, ciclopirox, clindamycin, clotrimazole, diiodohomatropine, diiodohydroxyquinoline, diiodohydroxyquinolone, diloxanide, eflornithine, ergometrine, ethylstibamine, etofamide, fenticonazole, fluconazole, fumagillin, furazolidone, hachimycin, hydroxystilbamidine, lauroguadine, mebendazole, melarsoprol, mepartricin, miconazole, miltefosine, naftifine, nifuratel, nifuroxime, nifurtimox, nimorazole, nitazoxanide, omidazole, oxophenarsine, paromomycin, pentamidine, propamidine, puromycin, pyrimethamine, quinapyramine, quinfamide, secnidazole, stilbamidine, suraminsodium, tenonitrozole, terconazole, tinidazole, tryparsamide, and ureastibamine.

One desirable antiprotozoal agent for use in the methods, compositions, and kits of the invention is metronidazole.

Metronidazole eliminates bacteria and other microorganisms that cause infections of the reproductive system, gastrointestinal tract, skin, vagina, and other areas of the body. It is also an antirosacea agent.

Anti-infective Agents

The invention features methods, compositions, and kits that include an anti-infective agent (or analog thereof) and an NsIDI. Topical anti-infective agents are effective against Gram-negative and Gram-positive bacteria. Anti-infective agents include topical antibiotics, sulphonamides, antiseptics, and disinfectants.

Anti-infective agents which can be used in the combinations of the invention include, without limitation, 1-naphthyl salicylate, 8-quinolinol, acetic acid, acid fuchsine, acriflavine, acriflavinium chloride, alcohol, alkyl poly(aminoethyl) glycine, alkyldiaminoglycine, alkylpoly(aminoethyl)glycine, alkylpolyaminoethylglycine, alkypoly(aminoethyl)glycine, alprostadil, aluminum sulfate, amikacin, ammonium benzoate, ammonium mandelate, arctostaphylos uva-ursi, bacitracin, baptisia, bearberry, benzalkonium chloride, benzethonium chloride, benzododecinium bromide, benzododecinium chloride, benzoxonium chloride, benzoyl peroxide, benzydamine, bismuth iodide oxide, bismuth iodosubgallate, bismuth tribromophenate, bithionol, bronopol, cadexomer iodine, calendula officinalis, carfecillin, carvacrol, cefixime, cefotetan, ceftibuten, cetalkonium chloride, cetirizine, cetrimide, cetrimonium, cetrimonium bromide, cetylpyridinium chloride, chamomile, chloramphenicol, chlorhexidine, chlorocresol, chloroxine, chloroxylenol, chlortetracycline, cinoxacin, ciprofloxacin, clioquinol, clobetasol propionate, clotrimazole, dapsone, demeclocycline, didecyldimethylammonium chloride, dioxidine, dodicin, domiphen bromide, emepronium carrageenate, enoxacin, erythromycin, escherichia coli, ethacridine, farnesol, fenticlor, flavoxate, fosfomycin, fosfomycin trometamol, fradiomycin, framycetin, furazidin, furazolidone, fusidic acid, gatifloxacin, gentamicin, gentian violet, halquinol, hexachlorophene, hexamidine, hexetidine, hyaluronic acid, hydrargaphen, hydrogen peroxide, ichthammol, iodinated alcohol, iodine, iodine monochloride, iodine piron, iodine trichloride, iodochlorhydroxyquin, iodoform, irgasan, isopropyl alcohol, isothiazole, kollodium, lactic acid, lapirium, mafenide, magnesium salicylate, melaleuca oil, merbromin, mercuric chloride, mesna, methanamine mandelate, methenamine, methionine, methylrosanilinium chloride, methylthioninium chloride, metiolato, metronidazole, monoethanolamide, mupirocin, nalidixic acid, neomycin, nidroxyzone, nifuroxazide, nifuroxime, nifurzide, nitrofural, nitrofurantoin, nitrofurazone, nitroxoline, norfloxacin, octenidine, ofloxacin, oil of sassafras, omidazole, oxolinic acid, oxychlorosene, pareira, pefloxacin, penicillin, pentosan polysulfate, pentoxifylline, phenazopyridine, phenoctide, phenol, phenosept, phenoxyethanol, pipemidic acid, piromidic acid, pivmecillinam, policresulen, polyvinox, povidone, povidone-iodine, propolis, pyrithione zinc, quinolone, resorcinol, rifampicin, rifamycin, rifamycin SV, rifaximin, rosoxacin, rufloxacin, salicylic acid, sodium dichloroisocyanurate, sodium dichromate(vi), sodium hypochlorate, sodium hypochloride, sodium hypochlorite, sodium sulfosuccinated undecenoic acid, sodium thiosulfate, sulfacarbamide, sulfadiazine, sulfadimidine, sulfamethizole, sulfamethoxypyridazine, sulfanilamide, sulfathiazole, sulfur, symclosene, tea tree oil, temafloxacin, terodiline, tetracycline, tevenel, thimerfonate sodium, thimerosal, thiram, timerosal, toloconium methylsulfate, tosylchloramide sodium, triclocarban, triclosan, trimethoprim, troclosene potassium, tyrothricin, vancomycin, and zinc oxide.

One desirable anti-infective agent for use in the methods, compositions, and kits of the invention is nitrofurazone.

Structural analogs of nitrofurazone which may be used in place of nitrofurazone in the combinations of the invention include, without limitation, 4-hydroxynitrofurazone, 5-nitro-2-furaldoxime, 5-nitrofurfurilidenaminoguanidine, guanofuracin, nidroxyzone, nifuraldezone, nifurethazone, nifuroxazid, nifuroxime, nifursemizone, nihydrazone, and nitrofuraldehyde diethylaminopropylsemicarbazone.

Sunscreen Agents

The invention features methods, compositions, and kits that include a sunscreen agent (or analog thereof) and an NsIDI.

Sunscreen agents are used to prevent sunburn. There are two kinds of sunscreen agents: chemical and physical. Chemical sunscreen agents protect skin from the sun by absorbing the ultraviolet (UV) and visible sun rays, while physical sunscreen agents reflect, scatter, absorb, or block these rays. Sunscreen agents often contain more than one ingredient. For example, products may contain one ingredient that provides protection against the ultraviolet A (UVA) sun rays and another ingredient that protects you from the ultraviolet B (UVB) sun rays, which are more likely to cause sunburn than the UVA sun rays. Ideally, coverage should include protection against both UVA and UVB sun rays.

Sunscreen agents which can be used in the combinations of the invention include, without limitation, avobenzone, dioxybenzone, homosalate, lisadimate, menthylanthranilate, minobenzoic acid, octocrylene, octylmethoxycinnamate, octylsalicylate, oxybenzone, padimate-o, phenylbenzimidazole, roxadimate, sulisobenzone, terephthalylidene dicamphor sulfonic acid, titaniumdioxide, trolaminesalicylate, and zinc oxide.

One desirable sunscreen agent for use in the methods, compositions, and kits of the invention is oxybenzone.

Structural analogs of oxybenzone which may be used in place of oxybenzone in the combinations of the invention include, without limitation, mexenone; 2,4-dihydroxybenzophenone; 4′-chloro-2-hydroxy-4-methoxybenzophenone; benzophenone 2′-hydroxy-5′-methoxy-; methanone, (2-hydroxy-4-methoxyphenyl)(4-methoxyphenyl); 4′-Fluoro-2-hydroxy-4-methoxybenzophenone; methanone, (2-hydroxy-4-(2-hydroxyethoxy)phenyl)phenyl-; benzophenone, 2-hydroxy-4-butoxy-; dioxybenzone; benzophenone, 2-hydroxy-4-methyl-; 2-hydroxy-4-methoxy-2′-methylbenzophenone; and 2-hydroxy-4-(2-phenoxyethoxy)benzophenone.

Humectants

The invention features methods, compositions, and kits that include a humectant (or analog thereof) and an NsIDI.

Humectants are substances that attract water when applied to the skin. The source of the water is transepidermal, unless the relative humidity is very high (>80%). Natural Moisturizing Factor (NMF) is a combination of several low molecular weight substances. These substances include amino acids, pyrrolidone carboxylic acid, lactate, urea, ammonia, uric acid, glucosamine, creatinine, citrate, sodium, potassium, calcium, magnesium, phosphate, organic acids, peptides, and other unidentified substances. Many of these substances are added to moisturizers to enhance its hygroscopic properties.

Humectants which can be used in the combinations of the invention include, without limitation, 1,3-di-6-quinolylurea, 1-butyl-3-metanilylurea, 4-nitrophenyl)urea, allylurea, alpha hydroxy acids, aluminum hexaurea sulfate triiodide, ammonium lactate, benzylurea, diazolidinyl urea, ectylurea, ethylene thiourea, glycerin, hydroxyurea, imidurea, inaidazolidinyl urea, isosorbide, lactate salts, maidazolidinyl urea, mannitol, mecloralurea, n,n′-dimethylthiourea, natural moisturizing factor (nmf), n-ethyl-n-nitrosourea, nitrourea, oxymethurea, pantothenol, phenylthiourea, phenylurea, sorbitol, sulfanilylurea, sulfathiourea, sym-diphenylthiourea, tetramethylurea, thiourea, urea, urea nitrate, urea stibamine, and ureaform.

One desirable humectant for use in the methods, compositions, and kits of the invention is urea.

Structural analogs of urea which may be used in place of urea in the combinations of the invention include, without limitation, polyurea, methylurea, and urea hydrochloride.

Vitamin D Compounds

The invention features methods, compositions, and kits that include a vitamin D compound and an NsIDI.

Vitamin D is a fat-soluble vitamin which plays an important role in regulating calcium, phosphorus and minerals in the body and for promoting normal bone development. The principal biologic function of vitamin D is to maintain serum calcium and phosphorus concentrations within the normal range by enhancing the efficiency of the small intestine to absorb these minerals from the diet.

Vitamin D is synthesized in the skin and under ideal conditions is not required in the diet. Its active form binds to specific receptors in target tissues resulting ultimately in an increased concentration of plasma Ca²⁺. Both dietary and intrinsically synthesized vitamin D, require activation to become biologically active.

As used herein, “vitamin D compounds” means vitamin D, antihypocalcemic agents and antihypoparathyroid agents. These can include, becocalcidiol, calcifediol (calderol), calcipotriene (Dovonex/Divonex, Dovobet/Divobet), calcipotriol, cholecalciferol calcitriol (rocaltrol), dihydrotachysterol (hytakerol), ergocalciferol (drisdol), mexacalcitol, tacalcitol, vitamin D2, vitamin D3 and the following analogs that are currently in clinical use: Rocaltrol® (Roche Laboratories), Calcijex® injectable calcitriol, investigational drugs from Leo Pharmaceutical including EB 1089 (24a,26a,27a-trihomo-22,24-diene-1α, 25-(OH)₂-D₃), KH 1060 (20-epi-22-oxa-24a,26a,27a-trihomo-1α,25-(OH)₂-D₃), MC 1288 and MC 903 (calcipotriol); Roche Pharmaceutical agents, such as 1,25-(OH)₂-16-ene-D₃, 1,25-(OH)₂-16-ene-23-yne-D₃, and 25-(OH)₂-16-ene-23-yne-D₃; Chugai Pharmaceuticals agents, such as 22-oxacalcitriol (22-oxa-1α,25-(OH)₂-D₃; 1α-(OH)D₅ from the University of Illinois; and drugs from the Institute of Medical Chemistry-Schering AG, such as ZK 161422 and ZK 157202. Any of the above-mentioned vitamin D compounds can be used in the combinations of the invention.

Zinc Salts

The invention features methods, compositions, and kits that include a zinc salt and an NsIDI.

Zinc salts which can be used in the combinations of the invention include, without limitation, aluminum zinc sulfate, bacitracin zinc, pentetate zinc trisodium, polaprezinc, potassium zinc sulfate, zinc acetate, zinc bromide, zinc caprylate, zinc carbonate, zinc chloride, zinc chromate(vi) hydroxide, zinc citrate, zinc cyanide, zinc fluoride, zinc formate, zinc gluconate, zinc hexafluorosilicate, zinc iodate, zinc iodide, zinc iodide-starch, zinc lactate, zinc meta-arsenite, zinc nitrate, zinc nitride, zinc nitrite, zinc oleate, zinc ortho-arsenate, zinc oxalate, zinc oxide, zinc perchlorate, zinc permanganate, zinc peroxide, zinc phosphate, zinc p-phenolsulfonate, zinc propionate, zinc pyrithione, zinc pyrophosphate, zinc salicylate, zinc selenate, zinc selenide, zinc silicate, zinc stearate, zinc sulfate, zinc sulfide, zinc tannate, zinc tartrate, zinc telluride, zinc thiocyanate, zinc undecylenate, zinc valerate, zinc-protoporphyrin.

Zinc salts can be administered either systemically or topically. Zinc salts are available in caplets, syrup and tablets. Dosages typically range from 5 mg to 200 mg. Zinc salts have also been formulated for topical administration. For example, for the control of dandruff between 0.1 and 2.0% (w/w) pyrithione zinc is applied at least twice a week. Zinc salts are also present in many skin protective creams. For example when used as a cream to prevent skin irritation such as diaper rash, 10 to 40% (w/w) zinc oxide is used. Combinations of the invention can be formulated in a similar fashion.

Therapy

The invention features methods for suppressing secretion of proinflammatory cytokines as a means for treating an immunoinflammatory disorder, proliferative skin disease, organ transplant rejection, or graft versus host disease. The suppression of cytokine secretion is achieved by administering one or more Group A enhancers in combination with one or more NsIDIs. While the examples describe particular Group A enhancers and one or more NsIDIs, it is understood that a combination of multiple agents is often desirable. For example, methotrexate, hydroxychloroquine, and sulfasalazine are commonly administered for the treatment of rheumatoid arthritis. Additional therapies are described below.

Psoriasis

The methods, compositions, and kits of the invention may be used for the treatment of psoriasis. If desired, one or more antipsoriatic agents typically used to treat psoriasis may be used as a substitute for or in addition to an NSIDI in the methods, compositions, and kits of the invention. Such agents include biologics (e.g., alefacept, infliximab, adalimumab, efalizumab, etanercept, and CDP-870), small molecule immunomodulators (e.g., VX 702, SCIO 469, doramapimod, RO 30201195, SCIO 323, DPC 333, pralnacasan, mycophenolate, and merimepodib), vitamin D compounds (e.g., calcpotriene, calcipotriol), psoralens (e.g., methoxsalen), retinoids (e.g., acitretin, tazarotene), DMARDs (e.g., methotrexate), anthralin, topical corticosteroids (e.g., clobetasol, triamcinolone, betamethasone, hydrocortisone, halobetasol, diflorasone, mometasone, halcinonide, fluticasone), systemic corticosteroids (e.g., prednisone, dexamethasone) antihistamines (e.g., hydroxyzine, loratadine, cetirizine, diphenhydramine, cyproheptadine, fexofenadine), tricyclic antidepressants (e.g., doxepin) and emollients, ointments and lotions.

Atopic Dermatitis

The methods, compositions, and kits of the invention may be used for the treatment of atopic dermatitis. If desired, one or more atopic dermatitis agents typically used to treat atopic dermatitis may be used as a substitute for or in addition to an NsIDI in the methods, composition, and kits of the invention. Such agents include topical corticosteroids (e.g., clobetasol, triamcinolone, betamethasone, hydrocortisone, halobetasol, diflorasone, mometasone, halcinonide, fluticasone), systemic corticosteroids (e.g., prednisone, dexamethasone) antihistamines (e.g., hydroxyzine, loratadine, cetirizine, diphenhydramine, cyproheptadine, fexofenadine), tricyclic antidepressants (e.g., doxepin) and emollients, ointments and lotions.

Hand Dermatitis

The methods, compositions, and kits of the invention may be used for the treatment of hand dermatitis. If desired, one or more hand dermatitis agents typically used to treat hand dermatitis may be used as a substitute for or in addition to an NSIDI in the methods, composition, and kits of the invention. Such agents include topical and systemic topical corticosteroids (e.g., clobetasol, triamcinolone, betamethasone, hydrocortisone, halobetasol, diflorasone, mometasone, halcinonide, fluticasone), systemic corticosteroids (e.g., prednisone, dexamethasone) antihistamines (e.g., hydroxyzine, loratadine, cetirizine, diphenhydramine, cyproheptadine, fexofenadine), tricyclic antidepressants (e.g., doxepin) and emollients, ointments and lotions.

Actinic Keratosis

The methods, compositions, and kits of the invention may be used for the treatment of actinic keratosis. If desired, one or more hand dermatitis agents typically used to treat hand dermatitis may be used as a substitute for or in addition to an NSIDI in the methods, composition, and kits of the invention. Such agents include chemotherapeutic agents (e.g. 5-fluorouracil), immune-response modifiers (imiquimod), non-steroid inflammatory agents (e.g., diclofenac), topical retinoids (e.g., adapalene), and photodynamic therapy using topical aminolevulinic acid.

Basal Cell Carcinoma

The methods, compositions, and kits of the invention may be used for the treatment of basal cell carcinoma, a proliferative skin disorder. If desired, one or more basal cell carcinoma agents typically used to treat basal cell carcinoma may be used as a substitute for or in addition to an NSIDI in the methods, composition, and kits of the invention. Such agents include chemotherapeutic agents (e.g. 5-fluorouracil), and immune-response modifiers.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to an NsIDI in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline). Thus, in one embodiment, the invention features the combination of Group A enhancer and a bronchodilator, and methods of treating COPD therewith.

Inflammatory Bowel Disease

The methods, compositions, and kits of the invention may be used for the treatment of inflammatory bowel disease. If desired, one or more agents typically used to treat inflammatory bowel disease may be used as a substitute for or in addition to an NsIDI in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of Group A enhancer and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used as a substitute for or in addition to an NsIDI in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of Group A enhancer with any of the foregoing agents, and methods of treating rheumatoid arthritis therewith.

Asthma

The methods, compositions, and kits of the invention may be used for the treatment of asthma. If desired, one or more agents typically used to treat asthma may be used as a substitute for or in addition to an NsIDI in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of Group A enhancer and any of the foregoing agents, and methods of treating asthma therewith.

Administration

In particular embodiments of any of the methods of the invention, an NsIDI and the Group A enhancer are administered within 10 days of each other, within five days of each other, within twenty-four hours of each other, or simultaneously.

The compounds may be formulated together as a single composition, or may be formulated and administered separately. One or both compounds may be administered in a low dosage or in a high dosage, each of which is defined herein. It may be desirable to administer to the patient other compounds, such as a corticosteroid, a small molecule immunomodulator (e.g., VX 702, SCIO 469, doramapimod, RO 30201195, SCIO 323, DPC 333, pranalcasan, mycophenolate, and merimepodib), a humectant (e.g., urea or pantothenol), a zinc salt, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the invention are especially useful for the treatment of immunoinflammatory disorders in combination with other anti-cytokine agents or agents that modulate the immune response to positively effect disease, such as agents that influence cell adhesion, or biologics (i.e., agents that block the action of IL-6, IL-1, IL-2, IL-12, IL-15 or TNF (e.g., etanercept, adelimumab, infliximab, or CDP-870). In this example (that of agents blocking the effect of TNFα), the combination therapy reduces the production of cytokines, etanercept or infliximab act on the remaining fraction of inflammatory cytokines, providing enhanced treatment.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Formulation

The administration of a combination of the invention (e.g., an NsIDI/Group A enhancer combination) may be by any suitable means that results in suppression of proinflammatory cytokine levels at the target region. A compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near simultaneous administration of the agents. Such co-formulated compositions can include the NsIDI and Group A enhancer formulated together either in a unit dosage form (e.g., in the same pill, capsule, or tablet) or non-unit dosage form (e.g., cream, liquid, or powder). It is to be understood that, when referring to the formulation of “NsIDI/Group A enhancer combinations,” the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Topical Formulations

For the prophylaxis and/or treatment of inflammatory dermatoses, the combinations of the invention are, desirably, formulated for topical administration. Topical formulations which can be used with the combinations of the invention include, without limitation, creams, foams, pastes, lotions, gels, sticks, sprays, patches, and ointments.

The combination of the invention may be mixed under sterile conditions with a pharmaceutically-acceptable carrier, and with any preservatives, buffers, or propellants which may be required. Any conventional pharmacologically and cosmetically acceptable vehicles may be used. For example, the compounds may also be administered in liposomal formulations that allow compounds to enter the skin. Such liposomal formulations are described in U.S. Pat. Nos. 5,169,637; 5,000,958; 5,049,388; 4,975,282; 5,194,266; 5,023,087; 5,688,525; 5,874,104; 5,409,704; 5,552,155; 5,356,633; 5,032,582; 4,994,213; and PCT Publication No. WO 96/40061. Examples of other appropriate vehicles are described in U.S. Pat. No. 4,877,805 and EP Publication No. 0586106A1. Suitable vehicles of the invention may also include mineral oil, petrolatum, polydecene, stearic acid, isopropyl myristate, polyoxyl 40 stearate, stearyl alcohol, or vegetable oil.

The formulations can include various conventional colorants, fragrances, thickeners (e.g., xanthan gum), preservatives, emollients (e.g., hydrocarbon oils, waxes, or silicones), demulcents, solubilizing excipients, dispersants, penetration enhancers, plasticizing agents, preservatives, stabilizers, demulsifiers, wetting agents, emulsifiers, moisturizers, astringents, deodorants, and the like can be added to provide additional benefits and improve the feel and/or appearance of the topical preparation.

Where the NsIDI or Group A enhancer has poor solubility in water at physiological pH, one or more solubilizing excipients may be a necessary component in the topical formulations.

Solubilization is taken to mean an improvement in the solubility by virtue of surface-active compounds that can convert substances that are insoluble or virtually insoluble in water into clear, or opalescent, aqueous solutions without changing the chemical structure of these substances in the process.

The solubilizates formed are notable for the fact that the substance is present in dissolved form in the molecular associations, micelles, of the surface-active compounds, which form in aqueous solution. The resulting solutions appear optically clear to opalescent.

Solubilizing excipients that may be used in the formulations of the invention include, without limitation, compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, ionic surfactants, tocopherol esters, and sterol esters. Each of these classes of excipient are commercially available and well known to those in the field of formulations.

The ointments, pastes, creams and gels may contain, in addition to a combination of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a combination of the invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches can be used with the added advantage of providing controlled delivery of one or more active ingredients in the combination of the invention. For example, absorption enhancers can also be used to increase the flux of active ingredients across the skin. Furthermore, either providing a rate controlling membrane or dispersing the compound in a polymer matrix or gel can control the rate flux of active ingredients across the skin.

Controlled Release Formulations

Administration of an NsIDI/Group A enhancer combination of the invention in which one or both of the active agents is formulated for controlled release is useful where the NsIDI or the Group A enhancer, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount of that is therapeutically effective for cytokine suppression. Accordingly, a sustained release formulation may be used to avoid frequent dosing that may be required in order to sustain the plasma levels of both agents at a therapeutic level. For example, in preferable oral pharmaceutical compositions of the invention, half-life and mean residency times from 10 to 20 hours for one or both agents of the combination of the invention are observed.

Many strategies can be pursued to obtain controlled release in which the rate of release outweighs the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the NsIDI and/or the Group A enhancer are released at period intervals, the release could be simultaneous, or a delayed release of one of the agents of the combination can be affected, when the early release of one particular agent is preferred over the other.

Controlled release formulations may include a degradable or nondegradable polymer, hydrogel, organogel, or other physical construct that modifies the bioabsorption, half-life or biodegradation of the agent. The controlled release formulation can be a material that is painted or otherwise applied onto the afflicted site, either internally or externally. In one example, the invention provides a biodegradable bolus or implant that is surgically inserted at or near a site of interest (for example, proximal to an arthritic joint). In another example, the controlled release formulation implant can be inserted into an organ, such as in the lower intestine for the treatment inflammatory bowel disease.

Hydrogels can be used in controlled release formulations for the NsIDI/Group A enhancer combinations of the present invention. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylactic acid, polycaprolactone, polyorthoesters, polyanhydrides or polypeptides. The entire network can be gelled into a biodegradable network that can be used to entrap and homogeneously disperse NsIDI/Group A enhancer combinations of the invention for delivery at a controlled rate.

Chitosan and mixtures of chitosan with carboxymethylcellulose sodium (CMC—Na) have been used as vehicles for the sustained release of drugs, as described by Inouye et al., Drug Design and Delivery 1: 297-305, 1987. Mixtures of these compounds and agents of the NsIDI/Group A enhancer combinations of the invention, when compressed under 200 kg/cm², form a tablet from which the active agent is slowly released upon administration to a subject. The release profile can be changed by varying the ratios of chitosan, CMC—Na, and active agent(s). The tablets can also contain other additives, including lactose, CaHPO₄ dihydrate, sucrose, crystalline cellulose, or croscarmellose sodium. Several examples are given in Table 2.

TABLE 2

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄*2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose Na | | | | 110 | | | | | | | | |

Baichwal, in U.S. Pat. No. 6,245,356, describes a sustained release oral solid dosage forms that includes agglomerated particles of a therapeutically active medicament (for example, an NsIDI/Group A enhancer combination or component thereof of the present invention) in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well-known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the combinations of the invention, Baichwal and Staniforth in U.S. Pat. No. 5,135,757 describe a free-flowing slow release granulation for use as a pharmaceutical excipient that includes from about 20 to about 70 percent or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and from about 30 to about 80 percent by weight of an inert pharmaceutical filler (such as, for example, lactose, dextrose, sucrose, sorbitol, xylitol, fructose or mixtures thereof). After mixing the excipient with an NsIDI/Group A enhancer combination, or combination agent, of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the combinations of the invention, Shell, in U.S. Pat. No. 5,007,790, describe sustained-release oral drug-dosage forms that release a drug in solution at a rate controlled by the solubility of the drug. The dosage form comprises a tablet or capsule that includes a plurality of particles of a dispersion of a limited solubility drug in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug and leach it from the particles, assuring that drug reaches the stomach in the solution state which is less injurious to the stomach than solid-state drug. The programmed eventual dissolution of the polymer depends upon the nature of the polymer and the degree of crosslinking. The polymer is nonfibrillar and substantially water soluble in its uncrosslinked state, and the degree of crosslinking is sufficient to enable the polymer to remain insoluble for the desired time period, normally at least from about 4 hours to 8 hours up to 12 hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of the NsIDI/Group A enhancer combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500 to 1000 μm size range.

Slow-release formulations can include a coating which is not readily water-soluble but which is slowly attacked and removed by water, or through which water can slowly permeate. Thus, for example, the NsIDI/Group A enhancer combinations of the invention can be spray-coated with a solution of a binder under continuously fluidizing conditions, such as describe by Kitamori et al., U.S. Pat. No. 4,036,948. Examples of water-soluble binders include pregelatinized starch (e.g., pregelatinized corn starch, pregelatinized white potato starch), pregelatinized modified starch, water-soluble celluloses (e.g., hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, organic solvent-soluble binders, such as cellulose derivatives (e.g., cellulose acetate phthalate, hydroxypropylmethyl-cellulose phthalate, ethylcellulose).

Combinations of the invention, or a component thereof, with sustained release properties can also be formulated by spray drying techniques. Yet another form of sustained release NsIDI/Group A enhancer combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, for example, Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

Extended- and/or controlled-release formulations of Group A enhancers, can be prepared using methods known in the art. For example, controlled-release formulations are described in U.S. Pat. No. 5,422,123. Thus, a system for the controlled release of an active substance including (a) a deposit-core containing an effective amount of the active substance and having defined geometric form, and (b) a support-platform applied to the deposit-core, wherein the deposit-core contains at least the active substance, and at least one member selected from (1) a polymeric material which swells on contact with water or aqueous liquids and a gellable polymeric material wherein the ratio of the swellable polymeric material to the gellable polymeric material is in the range 1:9 to 9:1, and (2) a single polymeric material having both swelling and gelling properties, and wherein the support-platform is an elastic support, applied to the deposit-core so that it partially covers the surface of the deposit-core and follows changes due to hydration of the deposit-core and is slowly soluble and/or slowly gellable in aqueous fluids. The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

A controlled-release formulation of budesonide (3 mg capsules) for the treatment of inflammatory bowel disease is available from AstraZeneca (sold as “Entocort™”). To make low dose levels of active substance possible, the active substance is micronised, suitably mixed with known diluents, such as starch and lactose, and granulated with PVP (polyvinylpyrrolidone). Further, the granulate is laminated with a sustained release inner layer resistant to a pH of 6.8 and a sustained release outer layer resistant to a pH of 1.0. The inner layer is made of Eudragit® RL (copolymer of acrylic and methacrylic esters with a low content of quaternary ammonium groups) and the outer layer is made of Eudragit®L (anionic polymer synthesized from methacrylic acid and methacrylic acid methyl ester).

A bilayer tablet can be formulated for an NsIDI/Group A enhancer combination of the invention in which different custom granulations are made for each agent of the combination and the two agents are compressed on a bi-layer press to form a single tablet. For example, 12.5 mg, 25 mg, 37.5 mg, or 50 mg of acyclovir, a Group A enhancer, is formulated for a controlled release that results in a acyclovir t_{1/2} of 15 to 20 hours may be combined in the same tablet with cyclosporine, which is formulated such that the t_{1/2} approximates that of acyclovir. In addition to controlling the rate of cyclosporine release in vivo, an enteric or delayed release coat may be included that delays the start of drug release such that the Tₘₐₓ of cyclosporine approximates that of acyclovir.

Cyclodextrins are cyclic polysaccharides containing naturally occurring D(+)-glucopyranose units in an α-(1,4) linkage. Alpha-, beta- and gamma-cyclodextrins, which contain, respectively, six, seven or eight glucopyranose units, are most commonly used and suitable examples are described in WO91/11172, WO94/02518 and WO98/55148. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); Cserhati, Analytical Biochemistry 225: 328-32, 1995; Husain et al., Applied Spectroscopy 46: 652-8, 1992. Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules. U.S. Pat. No. 4,727,064 describes pharmaceutical preparations consisting of a drug with substantially low water solubility and an amorphous, water-soluble cyclodextrin-based mixture in which the drug forms an inclusion complex with the cyclodextrins of the mixture.

Formation of a drug-cyclodextrin complex can modify the drug's solubility, dissolution rate, bioavailability, and/or stability properties.

Sulfobutylether-β3-cyclodextrin (SBE-β-CD, commercially available from CyDex, Inc, Overland Park, Kans., USA and sold as CAPTISOL®) can also be used as an aid in the preparation of sustained-release formulations of agents of the combinations of the present invention. For example, a sustained-release tablet has been prepared that includes prednisolone and SBE-β-CD compressed in a hydroxypropyl methylcellulose matrix (see Rao et al., J. Pharm. Sci. 90: 807-16, 2001). In another example of the use of various cyclodextrins, EP 1109806 B1 describes cyclodextrin complexes of pharmaceutical compounds, where α-, β-, or γ-cyclodextrins, including eptakis(2-6-di-α-methyl)-β-cyclodextrin, (2,3,6-tri-O-methyl)-β-cyclodextrin, monosuccinyl eptakis(2,6-di-O-methyl)-β-cyclodextrin, or 2-hydroxypropyl-3-cyclodextrin] in anhydrous or hydrated form formed complex ratios of agent to cyclodextrin of from 1:0.25 to 1:20 can be obtained.

Polymeric cyclodextrins have also been prepared, as described in U.S. patent application Ser. Nos. 10/021,294 and 10/021,312. The cyclodextrin polymers so formed can be useful for formulating agents of the combinations of the present invention. These multifunctional polymeric cyclodextrins are commercially available from Insert Therapeutics, Inc., Pasadena, Calif., USA.

As an alternative to direct complexation with agents, cyclodextrins may be used as an auxiliary additive, e.g., as a carrier, diluent or solubiliser. Formulations that include cyclodextrins and other agents of the combinations of the present invention (i.e., an NsIDI or Group A enhancer) can be prepared by methods similar to the preparations of the cyclodextrin formulations described herein.

One or both components of an NsIDI/Group A enhancer combination of the invention, or mixtures of the two components together, can be incorporated into liposomal carriers for administration. The liposomal carriers are composed of three general types of vesicle-forming lipid components. The first includes vesicle-forming lipids which will form the bulk of the vesicle structure in the liposome. Generally, these vesicle-forming lipids include any amphipathic lipids having hydrophobic and polar head group moieties, and which (a) can form spontaneously into bilayer vesicles in water, as exemplified by phospholipids, or (b) are stably incorporated into lipid bilayers, with its hydrophobic moiety in contact with the interior, hydrophobic region of the bilayer membrane, and its polar head group moiety oriented toward the exterior, polar surface of the membrane.

The vesicle-forming lipids of this type are preferably ones having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. The above-described lipids and phospholipids whose acyl chains have a variety of degrees of saturation can be obtained commercially, or prepared according to published methods. Other lipids that can be included in the invention are glycolipids and sterols, such as cholesterol.

The second general component includes a vesicle-forming lipid which is derivatized with a polymer chain which will form the polymer layer in the composition. The vesicle-forming lipids which can be used as the second general vesicle-forming lipid component are any of those described for the first general vesicle-forming lipid component. Vesicle forming lipids with diacyl chains, such as phospholipids, are preferred. One exemplary phospholipid is phosphatidylethanolamine (PE), which provides a reactive amino group which is convenient for coupling to the activated polymers. An exemplary PE is distearyl PE (DSPE).

The excipients present in the formulations of the invention are present in amounts such that the carrier forms a clear, or opalescent, aqueous dispersion of the NsIDI, the Group A enhancer, or the NsIDI/Group A enhancer combination sequestered within the liposome. The relative amount of a surface-active excipient necessary for the preparation of liposomal or solid lipid nanoparticulate formulations is determined using known methodology. For example, liposomes may be prepared by a variety of techniques, such as those detailed in Szoka et al, Biochim. Biophys. Acta. 601:559 (1980). Multilamellar vesicles (MLVs) can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

Other established liposomal formulation techniques can be applied as needed. For example, the use of liposomes to facilitate cellular uptake is described in U.S. Pat. Nos. 4,897,355 and 4,394,448.

Additional Applications

The compounds of the invention can be employed in immunomodulatory or mechanistic assays to determine whether other combinations, or single agents, are as effective as the combination in inhibiting secretion or production of proinflammatory cytokines or modulating immune response using assays generally known in the art, examples of which are described herein. For example, candidate compounds may be combined with a Group A enhancer (or metabolite or analog therein) or a Group A enhancer and applied to stimulated PBMCs. After a suitable time, the cells are examined for cytokine secretion or production or other suitable immune response. The relative effects of the combinations versus each other, and versus the single agents are compared, and effective compounds and combinations are identified.

The combinations of the invention are also useful tools in elucidating mechanistic information about the biological pathways involved in inflammation. Such information can lead to the development of new combinations or single agents for inhibiting inflammation caused by proinflammatory cytokines. Methods known in the art to determine biological pathways can be used to determine the pathway, or network of pathways affected by contacting cells stimulated to produce proinflammatory cytokines with the compounds of the invention. Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

The following examples are to illustrate the invention. They are not meant to limit the invention in any way.

### EXAMPLE 1

### Assay for Proinflammatory Cytokine-Suppressing Activity

Compound dilution matrices were assayed for the suppression of IL-2 or TNFα, as described below.

IL-2

A 100 μL suspension of diluted human white blood cells contained within each well of a polystyrene 384-well plate (NalgeNunc) was stimulated to secrete IL-2 by treatment with a final concentration of 10 ng/mL phorbol 12-myristate 13-acetate (Sigma, P-1585) and 750 ng/mL ionomycin (Sigma, I-0634). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-IL-2 antibody (PharMingen, #555051). After a two-hour incubation, the plate was washed (Tecan PowerWasher 384) with PBS containing 0.1% Tween 20 and incubated for an additional one hour with another anti-IL-2 antibody that was biotin labeled (Endogen, M600B) and HRP coupled to strepavidin (PharMingen, #13047E). After the plate was washed with 0.1% Tween 20/PBS, an HRP-luminescent substrate was added to each well and light intensity measured using a LJL Analyst plate luminometer.

TNFα Phorbol 12-Myistate 13-Acetate Stimulation

The effects of test compound combinations on TNFα secretion were assayed in white blood cells from human buffy coat stimulated with phorbol 12-myistate 13-acetate as follows. Human white blood cells from buffy coat were diluted 1:50 in media (RPMI; Gibco BRL, #11875-085), 10% fetal bovine serum (Gibco BRL, #25140-097), 2% penicillin/streptomycin (Gibco BRL, #15140-122)) and 50 μL of the diluted white blood cells was placed in each well of the assay plate. Drugs were added to the indicated concentration. After 16-18 hours of incubation at 37° C. with 5% CO₂ in a humidified incubator, the plate was centrifuged and the supernatant transferred to a white opaque polystyrene 384-well plate (NalgeNunc, Maxisorb) coated with an anti-TNFα antibody (PharMingen, #551220). After a two-hour incubation, the plate was washed (Tecan Powerwasher 384) with PBS containing 0.1% Tween 20 and incubated for one additional hour with biotin labeled anti-TNFα antibody (PharMingen, #554511) and HRP coupled to streptavidin (PharMingen, #13047E). The plate was then washed again with 0.1% Tween 20/PBS. An HRP-luminescent substrate was added to each well, and the light intensity of each well was measured using a plate luminometer.

Percent Inhibition

The percent inhibition (% I) for each well was calculated using the following formula:
%I=[(avg. untreated wells−treated well)/(avg. untreated wells)]×100
The average untreated well value (avg. untreated wells) is the arithmetic mean of 40 wells from the same assay plate treated with vehicle alone. Negative inhibition values result from local variations in treated wells as compared to untreated wells.

### EXAMPLE 2

### Preparation of Compounds

Stock solutions containing NsIDI and a Group A enhancer were made in dimethylsulfoxide (DMSO) at a final concentration of between 0 and 40 μM. Master plates were prepared to contain dilutions of the stock solutions of the compounds described above. Master plates were sealed and stored at −20° C. until ready for use.

NsIDI and Group A Enhancer Stocks

The stock solution containing cyclosporin A was made at a concentration of 1.2 mg/ml in DMSO. The stock solution of tacrolimus was made at a concentration of 0.04 mg/ml in DMSO.

The stock solution containing acyclovir was made at a concentration of 10 mg/mL in DMSO. The stock solution containing clotrimazole was made at a concentration of 10 mg/mL in DMSO. The stock solution containing zinc was made at a concentration of 10 mg/mL in DMSO. The stock solution containing urea was made at a concentration of 10 mg/mL in DMSO. The stock solution containing oxybenzone was made at a concentration of 10 mg/mL in DMSO. The stock solution containing vitamin D was made at a concentration of 10 mg/mL in DMSO. The stock solution containing nitrofurazone was made at a concentration of 10 mg/mL in DMSO. The stock solution containing metronidazole was made at a concentration of 10 mg/mL in DMSO. The stock solution containing colchicine was made at a concentration of 10 mg/mL in DMSO. The stock solution containing triclosan was made at a concentration of 10 mg/mL in DMSO.

Master plates were prepared to contain dilutions of the stock solutions of the compounds described above.

The final single agent plates were generated by transferring 1 μL of stock solution from the specific master plate to a dilution plate containing 100 μL of media (RPMI; Gibco BRL, #11875-085), 10% fetal bovine serum (Gibco BRL, #25140-097), 2% Penicillin/Streptomycin (Gibco BRL, #15140-122)) using the Packard Mini-Trak liquid handler. This dilution plate was then mixed and a 5 μL aliquot transferred to the final assay plate, which had been pre-filled with 50 μL/well RPMI media containing the appropriate stimulant to activate IL-2 or TNFα secretion (see Example 1, supra).

### EXAMPLE 3

### The Combination of Tacrolimus and Acyclovir Reduces IL-2 Secretion In Vitro

IL-2 secretion was measured by ELISA as described above after stimulation with phorbol 12-myristate 13-acetate and ionomycin. The effect of varying concentrations of tacrolimus, acyclovir, and tacrolimus in combination with acyclovir was compared to control wells stimulated without tacrolimus or acyclovir. The results of this experiment are shown in Table 3, below. The effects of the agents alone and in combination are shown as percent inhibition of IL-2 secretion. The data below represents single agent and combination data from one experiment. Wells without numbers represent data artifacts, which have been omitted.

TABLE 3

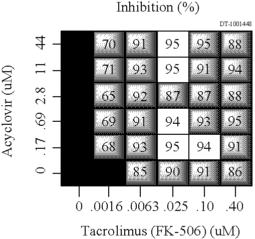

### EXAMPLE 4

### The Combination of Cyclosporine A and Acyclovir Reduces IL-2 Secretion In Vitro

IL-2 secretion was measured by ELISA as described above after stimulation with phorbol 12-myristate 13-acetate and ionomycin. The effect of varying concentrations of cyclosporine A, acyclovir, and cyclosporine A in combination with acyclovir was compared to control wells stimulated without cyclosporine A or acyclovir. The results of this experiment are shown in Table 4, below. The effects of the agents alone and in combination are shown as percent inhibition of IL-2 secretion. The data below represents single agent and combination data from one experiment.

TABLE 4

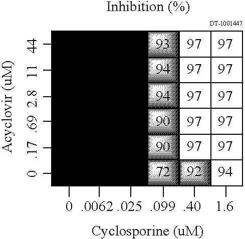

### EXAMPLE 5

### The Combination of Cyclosporine A and Clotrimazole Reduces IL-2 Secretion In Vitro

IL-2 secretion was measured by ELISA as described above after stimulation with phorbol 12-myristate 13-acetate and ionomycin. The effect of varying concentrations of cyclosporine A, clotrimazole and cyclosporine A in combination with clotrimazole was compared to control wells stimulated without cyclosporine A or clotrimazole. The results of this experiment are shown in Table 5, below. The effects of the agents alone and in combination are shown as percent inhibition of IL-2 secretion. The data below represents single agent and combination consensus data from five experiments. Wells without numbers represent data artifacts, which have been omitted.

TABLE 5

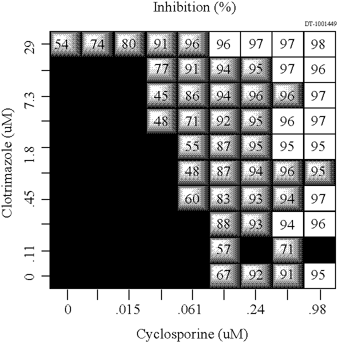

### EXAMPLE 6

### The Combination of Cyclosporine A and Clotrimazole Reduces TNFα Secretion In Vitro

TNFα secretion was measured by ELISA as described above after stimulation with phorbol 12-myristate 13-acetate and ionomycin. The effect of varying concentrations of cyclosporine A, clotrimazole and cyclosporine A in combination with clotrimazole was compared to control wells stimulated without cyclosporine A or clotrimazole. The results of this experiment are shown in Table 6, below. The effects of the agents alone and in combination are shown as percent inhibition of TNFα secretion. The data below represents single agent and combination consensus data from four experiments.

TABLE 6

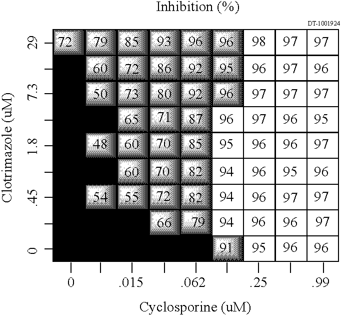

### EXAMPLE 7

### The Combination of Tacrolimus and Clotrimazole Reduces IL-2 Secretion In Vitro

IL-2 secretion was measured by ELISA as described above after stimulation with phorbol 12-myristate 13-acetate and ionomycin. The effect of varying concentrations of tacrolimus, clotrimazole, and tacrolimus in combination with clotrimazole was compared to control wells stimulated without tacrolimus or clotrimazole. The results of this experiment are shown in Table 7, below. The effects of the agents alone and in combination are shown as percent inhibition of IL-2 secretion. The data below represents single agent and combination consensus data from four experiments.

TABLE 7

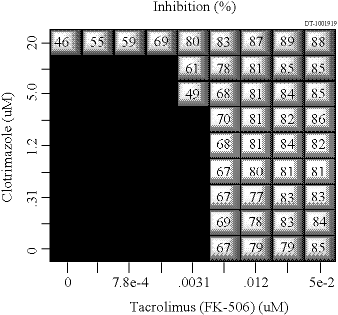

### EXAMPLE 8

### The Combination of Cyclosporine A and Colchicine Reduces IL-2 Secretion In Vitro

IL-2 secretion was measured by ELISA as described above after stimulation with phorbol 12-myristate 13-acetate and ionomycin. The effect of varying concentrations of cyclosporine A, colchicine, and cyclosporine A in combination with colchicine was compared to control wells stimulated without cyclosporine A or colchicine. The results of this experiment are shown in Table 8, below. The effects of the agents alone and in combination are shown as percent inhibition of IL-2 secretion. The data below represents single agent and combination data from one experiment.

TABLE 8

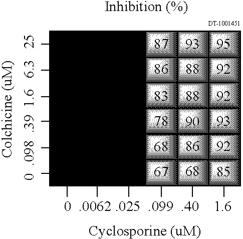

### EXAMPLE 9

### The Combination of Tacrolimus and Colchicine Reduces IL-2 Secretion In Vitro

IL-2 secretion was measured by ELISA as described above after stimulation with phorbol 12-myristate 13-acetate and ionomycin. The effect of varying concentrations of tacrolimus, colchicine, and tacrolimus in combination with colchicine was compared to control wells stimulated without tacrolimus or colchicine. The results of this experiment are shown in Table 9, below. The effects of the agents alone and in combination are shown as percent inhibition of IL-2 secretion. The data below represents single agent and combination data from one experiment.

TABLE 9

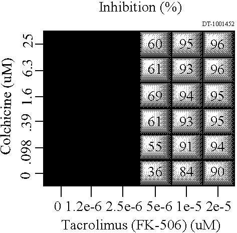

### EXAMPLE 10

### The Combination of Cyclosporine A and Metronidazole Reduces IL-2 Secretion In Vitro

IL-2 secretion was measured by ELISA as described above after stimulation with phorbol 12-myristate 13-acetate and ionomycin. The effect of varying concentrations of cyclosporine A, metronidazole, and cyclosporine A in combination with metronidazole was compared to control wells stimulated without cyclosporine A or metronidazole. The results of this experiment are shown in Table 10, below. The effects of the agents alone and in combination are shown as percent inhibition of IL-2 secretion. The data below represents single agent and combination data from one experiment.

TABLE 10

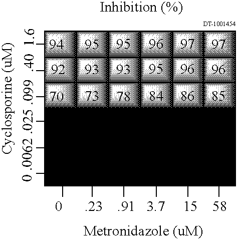

### EXAMPLE 11

### The Combination of Tacrolimus and Metronidazole Reduces IL-2 Secretion In Vitro

IL-2 secretion was measured by ELISA as described above after stimulation with phorbol 12-myristate 13-acetate and ionomycin. The effect of varying concentrations of tacrolimus, metronidazole, and tacrolimus in combination with metronidazole was compared to control wells stimulated without tacrolimus or metronidazole. The results of this experiment are shown in Table 11, below. The effects of the agents alone and in combination are shown as percent inhibition of IL-2 secretion. The data below represents single agent and combination data from one experiment.

TABLE 11

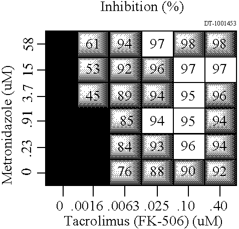

### EXAMPLE 12

### The Combination of Cyclosporine A and Nitrofurazone Reduces IL-2 Secretion In Vitro

IL-2 secretion was measured by ELISA as described above after stimulation with phorbol 12-myristate 13-acetate and ionomycin. The effect of varying concentrations of cyclosporine A, nitrofurazone, and cyclosporine A in combination with nitrofurazone was compared to control wells stimulated without cyclosporine A or nitrofurazone. The results of this experiment are shown in Table 12, below. The effects of the agents alone and in combination are shown as percent inhibition of IL-2 secretion. The data below represents single agent and combination data from one experiment.

TABLE 12

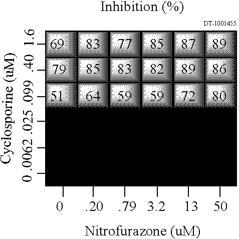

### EXAMPLE 13

### The Combination of Tacrolimus and Nitrofurazone Reduces IL-2 Secretion In Vitro

IL-2 secretion was measured by ELISA as described above after stimulation with phorbol 12-myristate 13-acetate and ionomycin. The effect of varying concentrations of tacrolimus, nitrofurazone, and tacrolimus in combination with nitrofurazone was compared to control wells stimulated without tacrolimus or nitrofurazone. The results of this experiment are shown in Table 13, below. The effects of the agents alone and in combination are shown as percent inhibition of IL-2 secretion. The data below represents single agent and combination data from one experiment.

TABLE 13

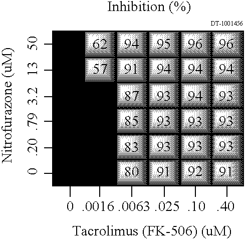

### EXAMPLE 14

### The Combination of Cyclosporine A and Oxybenzone Reduces IL-2 Secretion In Vitro

IL-2 secretion was measured by ELISA as described above after stimulation with phorbol 12-myristate 13-acetate and ionomycin. The effect of varying concentrations of cyclosporine A, oxybenzone, and cyclosporine A in combination with oxybenzone was compared to control wells stimulated without cyclosporine A or oxybenzone. The results of this experiment are shown in Table 14, below. The effects of the agents alone and in combination are shown as percent inhibition of IL-2 secretion. The data below represents single agent and combination data from one experiment.

TABLE 14

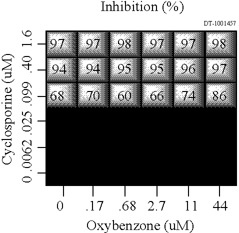

### EXAMPLE 15

### The Combination of Tacrolimus and Oxybenzone Reduces IL-2 Secretion In Vitro

IL-2 secretion was measured by ELISA as described above after stimulation with phorbol 12-myristate 13-acetate and ionomycin. The effect of varying concentrations of tacrolimus, oxybenzone, and tacrolimus in combination with oxybenzone was compared to control wells stimulated without tacrolimus or oxybenzone. The results of this experiment are shown in Table 15, below. The effects of the agents alone and in combination are shown as percent inhibition of IL-2 secretion. The data below represents single agent and combination data from one experiment.

TABLE 15

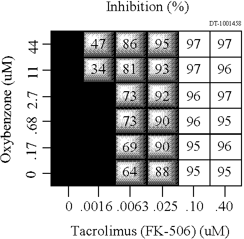

### EXAMPLE 16

### The Combination of Cyclosporine A and Urea Reduces IL-2 Secretion In Vitro

IL-2 secretion was measured by ELISA as described above after stimulation with phorbol 12-myristate 13-acetate and ionomycin. The effect of varying concentrations of cyclosporine A, urea, and cyclosporine A in combination with urea was compared to control wells stimulated without cyclosporine A or urea. The results of this experiment are shown in Table 16, below. The effects of the agents alone and in combination are shown as percent inhibition of IL-2 secretion. The data below represents single agent and combination data from one experiment.

TABLE 16

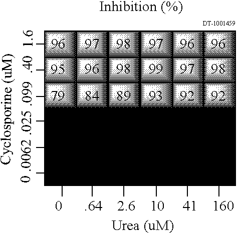

### EXAMPLE 17

### The Combination of Tacrolimus and Urea Reduces IL-2 Secretion In Vitro

IL-2 secretion was measured by ELISA as described above after stimulation with phorbol 12-myristate 13-acetate and ionomycin. The effect of varying concentrations of tacrolimus, urea, and tacrolimus in combination with urea was compared to control wells stimulated without tacrolimus or urea. The results of this experiment are shown in Table 17, below. The effects of the agents alone and in combination are shown as percent inhibition of IL-2 secretion. The data below represents single agent and combination data from one experiment. Wells without numbers represent data artifacts, which have been omitted.

TABLE 17

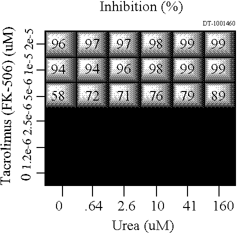

### EXAMPLE 18

### The Combination of Cyclosporine A and Vitamin D2 Reduces IL-2 Secretion In Vitro

IL-2 secretion was measured by ELISA as described above after stimulation with phorbol 12-myristate 13-acetate and ionomycin. The effect of varying concentrations of cyclosporine A, Vitamin D2, and cyclosporine A in combination with Vitamin D2 was compared to control wells stimulated without cyclosporine A or Vitamin D2. The results of this experiment are shown in Table 18, below. The effects of the agents alone and in combination are shown as percent inhibition of IL-2 secretion. The data below represents single agent and combination data from one experiment.

TABLE 18

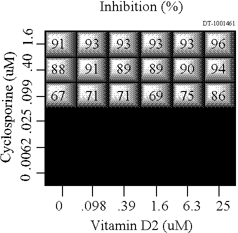

### EXAMPLE 19

### The Combination of Tacrolimus and Vitamin D2 Reduces IL-2 Secretion In Vitro

IL-2 secretion was measured by ELISA as described above after stimulation with phorbol 12-myristate 13-acetate and ionomycin. The effect of varying concentrations of tacrolimus, Vitamin D2, and tacrolimus in combination with Vitamin D2 was compared to control wells stimulated without tacrolimus or Vitamin D2. The results of this experiment are shown in Table 19, below. The effects of the agents alone and in combination are shown as percent inhibition of IL-2 secretion. The data below represents single agent and combination data from one experiment.

TABLE 19

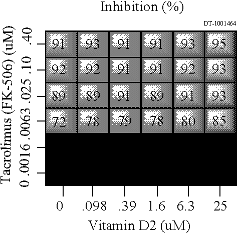

### EXAMPLE 20

### The Combination of Cyclosporine A and Vitamin D3 Reduces IL-2 Secretion In Vitro

IL-2 secretion was measured by ELISA as described above after stimulation with phorbol 12-myristate 13-acetate and ionomycin. The effect of varying concentrations of cyclosporine A, Vitamin D3, and cyclosporine A in combination with Vitamin D3 was compared to control wells stimulated without cyclosporine A or Vitamin D3. The results of this experiment are shown in Table 20, below. The effects of the agents alone and in combination are shown as percent inhibition of IL-2 secretion. The data below represents single agent and combination data from one experiment.

TABLE 20

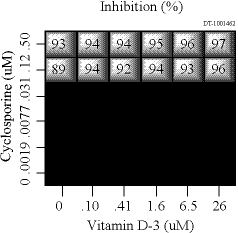

### EXAMPLE 21

### The Combination of Tacrolimus and Vitamin D3 Reduces IL-2 Secretion In Vitro

IL-2 secretion was measured by ELISA as described above after stimulation with phorbol 12-myristate 13-acetate and ionomycin. The effect of varying concentrations of tacrolimus, Vitamin D3, and tacrolimus in combination with Vitamin D3 was compared to control wells stimulated without tacrolimus or Vitamin D3. The results of this experiment are shown in Table 21, below. The effects of the agents alone and in combination are shown as percent inhibition of IL-2 secretion. The data below represents single agent and combination data from one experiment.

TABLE 21

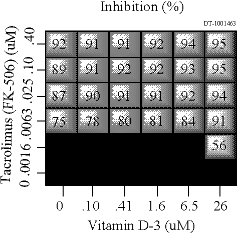

### EXAMPLE 22

### The Combination of Cyclosporine A and Zinc Acetate Reduces IL-2 Secretion In Vitro

IL-2 secretion was measured by ELISA as described above after stimulation with phorbol 12-myristate 13-acetate and ionomycin. The effect of varying concentrations of cyclosporine A, zinc acetate, and cyclosporine A in combination with zinc acetate was compared to control wells stimulated without cyclosporine A or zinc acetate. The results of this experiment are shown in Table 22, below. The effects of the agents alone and in combination are shown as percent inhibition of IL-2 secretion. The data below represents single agent and combination data from one experiment. Wells without numbers represent data artifacts, which have been omitted.

TABLE 22

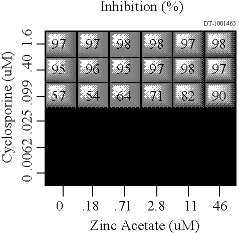

### EXAMPLE 23

### The Combination of Cyclosporine A and Zinc Chloride Reduces IL-2 Secretion In Vitro

IL-2 secretion was measured by ELISA as described above after stimulation with phorbol 12-myristate 13-acetate and ionomycin. The effect of varying concentrations of cyclosporine A, zinc chloride, and cyclosporine A in combination with zinc chloride was compared to control wells stimulated without cyclosporine A or zinc chloride. The results of this experiment are shown in Table 23, below. The effects of the agents alone and in combination are shown as percent inhibition of IL-2 secretion. The data below represents single agent and combination data from one experiment.

TABLE 23

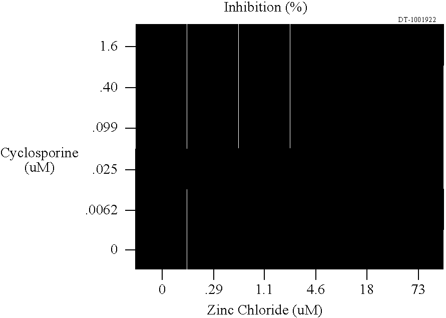

### EXAMPLE 24

### The Combination of Tacrolimus and Zinc Acetate Reduces IL-2 Secretion In Vitro

IL-2 secretion was measured by ELISA as described above after stimulation with phorbol 12-myristate 13-acetate and ionomycin. The effect of varying concentrations of tacrolimus, zinc acetate, and tacrolimus in combination with zinc acetate was compared to control wells stimulated without tacrolimus or zinc acetate. The results of this experiment are shown in Table 24, below. The effects of the agents alone and in combination are shown as percent inhibition of IL-2 secretion. The data below represents single agent and combination data from one experiment.

TABLE 24

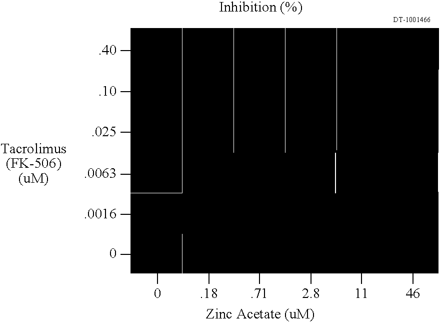

### EXAMPLE 25

### The Combination of Tacrolimus and Zinc Chloride Reduces IL-2 Secretion In Vitro

IL-2 secretion was measured by ELISA as described above after stimulation with phorbol 12-myristate 13-acetate and ionomycin. The effect of varying concentrations of tacrolimus, zinc chloride, and tacrolimus in combination with zinc chloride was compared to control wells stimulated without tacrolimus or zinc chloride. The results of this experiment are shown in Table 25, below. The effects of the agents alone and in combination are shown as percent inhibition of IL-2 secretion. The data below represents single agent and combination data from one experiment.

TABLE 25

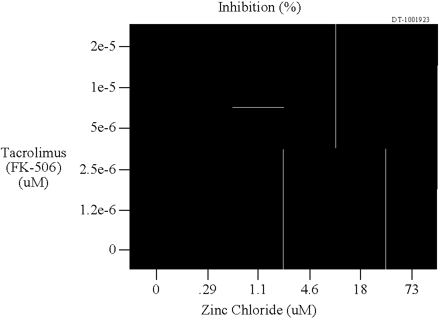

### OTHER EMBODIMENTS

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

What is claimed is:

1. A composition comprising a non-steroidal immunophilin-dependent immunosuppressant (NsIDI) and Group A enhancer in amounts that together are sufficient in vivo to decrease proinflammatory cytokine secretion or production or to treat an immunoinflammatory disorder.

2. The composition of claim 1, wherein said NsIDI is a calcineurin inhibitor.

3. The composition of claim 2, wherein said calcineurin inhibitor is cyclosporine, tacrolimus, ascomycin, pimecrolimus, ABT-281, or ISAtx247.

4. The composition of claim 1, wherein said NsIDI binds to FK506-binding protein.

5. The composition of claim 4, wherein said NsIDI is rapamycin or everolimus.

6. The composition of claim 1, wherein said Group A enhancer is an antiviral agent, antifungal agent, antigout agent, antiprotozoal agent, anti-infective agent, sunscreen agent, microtubule inhibitor, humectant, or zinc salt.

7. The composition of claim 6, wherein said Group A enhancer is an antiviral agent.

8. The composition of claim 7, wherein said antiviral agent is acyclovir.

9. The composition of claim 6, wherein said Group A enhancer is an antifungal agent.

10. The composition of claim 9, wherein said antifungal agent is clotrimazole.

11. The composition of claim 6, Group A enhancer is an antigout agent.

12. The composition of claim 11, wherein said antigout agent is colchicine.

13. The composition of claim 6, wherein said Group A enhancer is an antiprotozoal agent.

14. The composition of claim 13, wherein said antiprotozoal agent is metronidazole.

15. The composition of claim 6, wherein said Group A enhancer is an anti-infective agent.

16. The composition of claim 15, wherein said anti-infective agent is nitrofurazone.

17. The composition of claim 6, wherein said Group A enhancer is a sunscreen agent.

18. The composition of claim 17, wherein said sunscreen agent is oxybenzone.

19. The composition of claim 6, wherein said Group A enhancer is a humectant.

20. The composition of claim 19, wherein said humectant is urea.

21. The composition of claim 6, wherein said Group A enhancer is a microtubule inhibitor.

22. The composition of claim 6, wherein said Group A enhancer is a zinc salt.

23. The composition of claim 1, wherein said composition is formulated for topical administration.

24. The composition of claim 23, wherein said composition comprises greater than 1.0% (w/w) zinc.

25. The composition of claim 23, wherein said composition is formulated as a cream, foam, paste, lotion, gel, stick, spray, patch, or ointment.

26. The composition of claim 1, wherein said composition is formulated for systemic administration.

27. The composition of claim 1, wherein said composition further comprises an additional agent selected from a non-steroidal anti-inflammatory drug (NSAID), COX-2 inhibitor, biologic, small molecule immunomodulator, disease-modifying anti-rheumatic drugs (DMARD), xanthine, anticholinergic compound, beta receptor agonist, bronchodilator, corticosteroid, humectant, zinc salt, vitamin D compound, psoralen, retinoid, and 5-amino salicylic acid.

28. The composition of claim 27, wherein said additional agent is an NSAID selected from ibuprofen, diclofenac, and naproxen.

29. The composition of claim 27, wherein said additional agent is a COX-2 inhibitor selected from rofecoxib, celecoxib, valdecoxib, and lumiracoxib.

30. The composition of claim 27, wherein said additional agent is a biologic selected from adelimumab, etanercept, and infliximab.

31. The composition of claim 27, wherein said additional agent is a DMARD selected from methotrexate and leflunomide.

32. The composition of claim 27, wherein said additional agent is a xanthine selected from theophylline.

33. The composition of claim 27, wherein said additional agent is an anticholinergic compound selected from ipratropium and tiotropium.

34. The composition of claim 27, wherein said additional agent is a beta receptor agonist selected from ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline.

35. The composition of claim 27, wherein said additional agent is a vitamin D compound selected from calcipotriene and calcipotriol.

36. The composition of claim 27, wherein said additional agent is a psoralen selected from methoxsalen.

37. The composition of claim 27, wherein said additional agent is a retinoid selected from acitretin and tazoretene.

38. The composition of claim 27, wherein said additional agent is a 5-amino salicylic acid selected from mesalamine, sulfasalazine, balsalazide disodium, and olsalazine sodium.

39. The composition of claim 27, wherein said additional agent is a small molecule immunomodulator selected from VX 702, SCIO 469, doramapimod, RO 30201195, SCIO 323, DPC 333, pranalcasan, mycophenolate, and merimepodib.

40. The composition of claim 27, wherein said additional agent is a humectant selected from urea and pantothenol.

41. The composition of claim 27, wherein said additional agent is a zinc salt.

42. The composition of claim 27, wherein said additional agent is a corticosteroid selected from clobetasol, triamcinolone, betamethasone, hydrocortisone, halobetasol, diflorasone, mometasone, halcinonide, fluticasone, prednisone, and dexamethasone.

43. A method of decreasing proinflammatory cytokine secretion or production in a patient, said method comprising administering to the patient an NsIDI and a Group A enhancer simultaneously or within 14 days of each other in amounts sufficient in vivo to decrease proinflammatory cytokine secretion or production in said patient.

44. A method for treating a patient diagnosed with or at risk of developing an immunoinflammatory disorder, said method comprising administering to the patient an NsIDI and a Group A enhancer simultaneously or within 14 days of each other in amounts sufficient to treat said patient.

45. The method of claim 44, wherein said immunoinflammatory disorder is a dermal inflammatory disorder, rheumatoid arthritis, Crohn's disease, ulcerative colitis, asthma, chronic obstructive pulmonary disease, polymylagia rheumatica, giant cell arteritis, systemic lupus erythematosus, multiple sclerosis, myasthenia gravis, ankylosing spondylitis, or psoriatic arthritis.

46. The method of claim 44, wherein said NsIDI is cyclosporine, tacrolimus, ascomycin, pimecrolimus, ABT-281, ISAtx247, rapamycin, or everolimus.

47. The method of claim 44, where said Group A enhancer is an antiviral agent, antifungal agent, antigout agent, antiprotozoal agent, anti-infective agent, sunscreen agent, microtubule inhibitor, humectant, or zinc salt.

48. The method of claim 44, wherein said Group A enhancer is an antiviral agent.

49. The method of claim 48, wherein said antiviral agent is acyclovir.

50. The method of claim 44, wherein said Group A enhancer is an antifungal agent.

51. The method of claim 50, wherein said antifungal agent is clotrimazole.

52. The method of claim 44, Group A enhancer is an antigout agent.

53. The method of claim 52, wherein said antigout agent is colchicine.

54. The method of claim 44, wherein said Group A enhancer is an antiprotozoal agent.

55. The method of claim 54, wherein said antiprotozoal agent is metronidazole.

56. The method of claim 44, wherein said Group A enhancer is an anti-infective agent.

57. The method of claim 56, wherein said anti-infective agent is nitrofurazone.

58. The method of claim 44, wherein said Group A enhancer is a sunscreen agent.

59. The method of claim 58, wherein said sunscreen agent is oxybenzone.

60. The method of claim 44, wherein said Group A enhancer is a humectant.

61. The method of claim 60, wherein said humectant is urea.

62. The method of claim 44, wherein said Group A enhancer is a microtubule inhibitor.

63. The method of claim 44, wherein said Group A enhancer is a zinc salt.

64. The method of claim 44, wherein said NsIDI and said Group A enhancer are administered topically.

65. The method of claim 45, wherein said immunoinflammatory disorder is a dermal inflammatory disorder.

66. The method of claim 65, wherein said dermal inflammatory disorder is psoriasis, atopic dermatitis, hand dermatitis, or actinic keratosis.

67. The method of claim 44, wherein said NsIDI and said Group A enhancer are administered systemically.

68. The method of claim 44, wherein said method further comprises administering an additional agent selected from an NSAID, COX-2 inhibitor, small molecule immunomodulator, disease-modifying anti-rheumatic drugs (DMARD), xanthine, anticholinergic compound, beta receptor agonist, bronchodilator, corticosteroid, humectant, zinc salt, vitamin D compound, psoralen, retinoid, and 5-amino salicylic acid.

69. The method of claim 68, wherein said method comprises administering to said patient a composition of any of claims 28-42.

70. A method of decreasing proinflammatory cytokine secretion or production in a cell, said method comprising contacting said cell with an NsIDI and a Group A enhancer simultaneously or within 14 days of each other in amounts sufficient in vivo to decrease proinflammatory cytokine secretion or production in said cell.

71. The method of claim 70, wherein said cell is a mammalian cell in vivo.

72. A method for treating a patient diagnosed with or at risk of developing proliferative skin disease, said method comprising administering to the patient an NsIDI and a Group A enhancer simultaneously or within 14 days of each other in amounts sufficient to treat said patient.

73. A kit, comprising:
(i) a composition comprising an NsIDI and a Group A enhancer; and
(ii) instructions for administering said composition to a patient diagnosed with or at risk of developing an immunoinflammatory disorder.

74. A kit, comprising:
(i) an NsIDI;
(ii) a Group A enhancer; and
(iii) instructions for administering said NsIDI and said Group A enhancer to a patient diagnosed with or at risk of developing an immunoinflammatory disorder.

75. A kit comprising:
(i) an NsIDI; and
(ii) instructions for administering said NsIDI and a Group A enhancer to a patient diagnosed with or at risk of developing an immunoinflammatory disorder.

76. A kit comprising:
(i) a Group A enhancer; and
(ii) instructions for administering said Group A enhancer and an NsIDI to a patient diagnosed with or at risk of developing an immunoinflammatory disorder.
